(19) United States
(12) Patent Application Publication
(10) Pub. No.: US 2010/0041706 A1
(43) Pub. Date: Feb. 18, 2010

(54) Compounds 501

(75) Inventors: Hans Âström, Sodertalje (SE); Veronica Profir, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 12/539,082

(22) Filed: Aug. 11, 2009

Related U.S. Application Data
(60) Provisional application No. 61/088,053, filed on Aug. 12, 2008.

(51) Int. Cl. A61K 31/4439; C07D 413/14

(52) U.S. Cl. 514/340; 546/272.1

(57) ABSTRACT

The present invention relates to a novel crystalline form of 4-(5-{(1R)-1-[5-(3-chlorophenyl)isoxazol-3-yl]ethoxy}-4-methyl-4H-1,2,4-triazol-3-yl)pyridine. Further, the present invention also relates to the use of the novel crystalline form for the treatment of gastrointestinal disorders, pharmaceutical compositions containing it as well as processes for the preparation of the novel crystalline form.

### FIELD OF THE INVENTION

The present invention relates to a novel crystalline form (modification B) of 4-(5-{(1R)-1-[5-(3-chlorophenyl)isoxazol-3-yl]ethoxy}-4-methyl-4H-1,2,4-triazol-3-yl)pyridine possessing favourable characteristics. Further, the present invention also relates to the use of the novel crystalline form for prevention or treatment of a mGluR5 receptor-mediated disorder, such as a neurological, psychiatric or a gastrointestinal disorder. The invention also provides pharmaceutical compositions containing it as well as processes for the preparation of the novel crystalline form.

### BACKGROUND OF THE INVENTION

The compound 4-(5-{(1R)-1-[5-(3-chlorophenyl)isoxazol-3-yl]ethoxy}-4-methyl-4H-1,2,4-triazol-3-yl)pyridine is described in WO2007/040982.

### BRIEF DESCRIPTION OF THE DRAWINGS

FIG. 1 is an X-ray powder diffractogram of 4-(5-{(1R)-1-[5-(3-chlorophenyl)isoxazol-3-yl]ethoxy}-4-methyl-4H-1,2,4-triazol-3-yl)pyridine, modification B.

### DESCRIPTION OF THE INVENTION

It has been found that 4-(5-{(1R)-1-[5-(3-chlorophenyl)isoxazol-3-yl]ethoxy}-4-methyl-4H-1,2,4-triazol-3-yl)pyridine can exist in a novel crystalline form possessing favourable characteristics. The novel crystal form for the first time disclosed is hereinafter referred to as 4-(5-{(1R)-1-[5-(3-chlorophenyl)isoxazol-3-yl]ethoxy}-4-methyl-4H-1,2,4-triazol-3-yl)pyridine, modification B. The novel crystalline form can be characterized by its X-ray powder diffraction pattern, and in particular its d-spacing values of 7.6 Å and 5.6 Å.

It is thus an object of the present invention to provide a crystalline form of the neutral form of 4-(5-{(1R)-1-[5-(3-chlorophenyl)isoxazol-3-yl]ethoxy}-4-methyl-4H-1,2,4-triazol-3-yl)pyridine with advantageous properties.

It is an aspect of the present invention to provide 4-(5-{(1R)-1-[5-(3-chlorophenyl)isoxazol-3-yl]ethoxy}-4-methyl-4H-1,2,4-triazol-3-yl)pyridine, modification B.

4-(5-{(1R)-1-[5-(3-chlorophenyl)isoxazol-3-yl]ethoxy}-4-methyl-4H-1,2,4-triazol-3-yl)pyridine, modification B, is characterized in providing an X-ray powder diffraction pattern, exhibiting substantially the following main peaks with d-values (d-value: the spacing between successive parallel hkl planes in a crystal lattice):

| d-spacing value (Å) | Relative intensity |
|---|---|
| 10.7 | Very weak |
| 7.6 | Very strong |
| 6.8 | Medium |
| 5.6 | Very strong |
| 4.15 | Strong |
| 4.11 | Medium |
| 3.83 | Medium |
| 3.76 | Medium |
| 3.58 | Weak |

The peaks, identified with d-values calculated from the Bragg formula and intensities, have been extracted from the diffractogram of 4-(5-{(1R)-1-[5-(3-chlorophenyl)isoxazol-3-yl]ethoxy}-4-methyl-4H-1,2,4-triazol-3-yl)pyridine, modification B. Only the main peaks, that are the most characteristic, significant, distinct and/or reproducible, have been tabulated (a number of weak peaks have been omitted. Peaks are only listed up to 35 degrees 2θ), but additional peaks can be extracted, using conventional methods, from the diffractogram. The presence of these main peaks, reproducible and within the error limit, is for most circumstances sufficient to establish the presence of said crystal modification B.

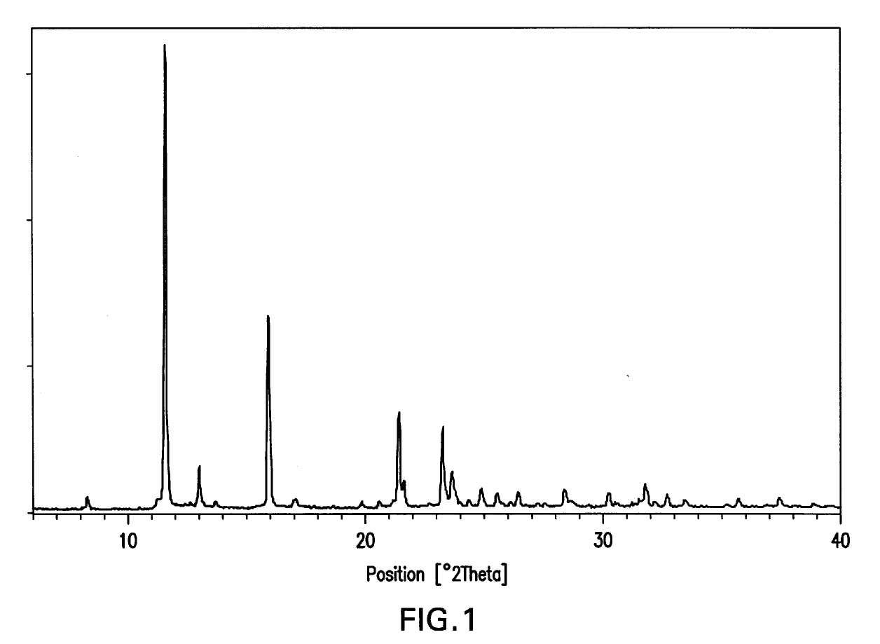

4-(5-{(1R)-1-[5-(3-chlorophenyl)isoxazol-3-yl]ethoxy}-4-methyl-4H-1,2,4-triazol-3-yl)pyridine, modification B, is further characterized by an X-ray powder diffraction pattern essentially as shown in FIG. 1.

4-(5-{(1R)-1-[5-(3-chlorophenyl)isoxazol-3-yl]ethoxy}-4-methyl-4H-1,2,4-triazol-3-yl)pyridine, modification B, is a crystalline form exhibiting advantageous properties over the amorphous form, such as increased chemical and physical stability, lower hygroscopicity, higher purity, better yield and robust handling properties during manufacturing and post processing.

In one embodiment the invention provides a process for the preparation of 4-(5-{(1R)-1-[5-(3-chlorophenyl)isoxazol-3-yl]ethoxy}-4-methyl-4H-1,2,4-triazol-3-yl)pyridine, modification B.

In one embodiment, the invention provides a process for preparing crystalline 4-(5-{(1R)-1-[5-(3-chlorophenyl)isoxazol-3-yl]ethoxy}-4-methyl-4H-1,2,4-triazol-3-yl)pyridine comprising the steps of:

a) mixing (R)-1-[5-(3-chloro-phenyl)-isoxazol-3-yl]-ethanol, 4-(5-methanesulfonyl-4-methyl-4H-[1,2,4]triazol-3-yl) pyridine and a base in a non-aqueous polar solvent;

b) heating the mixture to at least 60° C. for at least 10 hours;

c) cooling the reaction mixture to a temperature of at most 25° C.; and

d) adding water to the cooled reaction mixture, optionally together with crystalline 4-(5-{(1R)-1-[5-(3-chlorophenyl)isoxazol-3-yl]ethoxy}-4-methyl-4H-1,2,4-triazol-3-yl)pyridine according to claim 2, as seed crystals.

In one embodiment, the non-aqueous polar solvent is selected from the group of dimethylsulfoxide, dimethylformamide, N-methyl pyrrolidone and acetonitrile.

Alternatively, alcohols (e.g. methanol, ethanol, n-propanol, 2-propanol, n-butanol, tert-butanol) could be used as single crystallization solvent or in any combination with or without water as co-solvent. Furthermore, esters (e.g. ethyl acetate, n-butyl acetate, isopropyl acetate), ethers (e.g. methyl tert-butyl ether, tetrahydrofurane, 2-methyl tetrahydrofurane 1,4-dioxane) or ketones (e.g. acetone, methylethyl ketone, methyl iso-butyl ketone) may be considered as single crystallization solvent or any combination.

In one embodiment, the base is selected from the group of caesium carbonate and potassium tert-butoxide.

In another embodiment, the invention provides a process for preparing crystalline 4-(5-{(1R)-1-[5-(3-chlorophenyl)isoxazol-3-yl]ethoxy}-4-methyl-4H-1,2,4-triazol-3-yl)pyridine, modification B, wherein crystalline or amorphous 4-(5-{(1R)-1-[5-(3-chlorophenyl)isoxazol-3-yl]ethoxy}-4-methyl-4H-1,2,4-triazol-3-yl)pyridine is suspended in a solvent chosen from the group of ethyl acetate or 2-propanol at a temperature of at most 20° C. for at least 1 h.

4-(5-{(1R)-1-[5-(3-chlorophenyl)isoxazol-3-yl]ethoxy}-4-methyl-4H-1,2,4-triazol-3-yl)pyridine, modification B, obtained according to the present invention is substantially free from other crystal and non-crystal forms of 4-(5-{(1R)-1-[5-(3-chlorophenyl)isoxazol-3-yl]ethoxy}-4-methyl-4H-1,2,4-triazol-3-yl)pyridine. The term “substantially free from other crystal and non-crystal forms of 4-(5-{(1R)-1-[5-(3-chlorophenyl)isoxazol-3-yl]ethoxy}-4-methyl-4H-1,2,4-triazol-3-yl)pyridine” shall be understood to mean that the desired crystal form of 4-(5-{(1R)-1-[5-(3-chlorophenyl)isoxazol-3-yl]ethoxy}-4-methyl-4H-1,2,4-triazol-3-yl)pyridine contains less than 15%, preferably less than 10%, more preferably less than 5% of any other forms of 4-(5-{(1R)-1-[5-(3-chlorophenyl)isoxazol-3-yl]ethoxy}-4-methyl-4H-1,2,4-triazol-3-yl)pyridine.

The crystal modification B according to the present invention is useful for the prevention or treatment of gastroesophageal reflux disease, IBS, functional dyspepsia, cough, obesity, Alzheimer's disease, senile dementia, AIDS-induced dementia, Parkinson's disease, amyotrophic lateral sclerosis, Huntington's Chorea, migraine, epilepsy, schizophrenia, depression, anxiety, acute anxiety, obsessive compulsive disorder, ophtalmological disorders such as retinopathies, diabetic retinopathies, glaucoma, auditory neuropathic disorders such as tinnitus, chemotherapy-induced neuropathies, post-herpetic neuralgia and trigeminal neuralgia, tolerance, dependency, addiction and craving disorders, neurodevelopmental disorders including Fragile X, autism, mental retardation, schizophrenia and Down's Syndrome, pain related to migraine, inflammatory pain, chronic pain disorders, acute pain disorders, neuropathic pain disorders such as diabetic neuropathies, arthritis and rheumatitiod diseases, low back pain, post-operative pain, pain associated with various conditions including angina, renal or billiary colic, menstruation, migraine and gout, stroke, head trauma, anoxic and ischemic injuries, hypoglycemia, cardiovascular diseases and epilepsy.

It is further provided a pharmaceutical composition comprising the crystal modification B according to the present invention, as active ingredient, in association with a pharmaceutically acceptable carrier, diluent or excipient and optionally other active pharmaceutical ingredients. The pharmaceutical compositions of this invention may be administered in standard manner for the disease condition that it is desired to treat, for example by oral, topical, parenteral, buccal, nasal, vaginal or rectal administration or by inhalation or insufflation. For these purposes the crystal modification B according to the present invention may be formulated by means known in the art into the form of, for example, tablets, pellets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

In addition to the crystal modification B according to the present invention, the pharmaceutical composition of this invention may also contain, or be co-administered (simultaneously or sequentially) with, one or more pharmacological agents of value in treating one or more disease conditions referred to herein.

Suitable daily doses of the compounds of formula I in the treatment of a mammal, including man are approximately 0.01 to 250 mg/kg bodyweight at peroral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

In the practice of the invention, the most suitable route of administration as well as the therapeutic dose will depend on the nature and severity of the disease to be treated. The dose, and dose frequency, may also vary according to the age, body weight and response of the individual patient.

The crystal modification B according to the present invention may be further processed before formulation into a suitable pharmaceutical formulation. For example, the crystal modification B may be milled or ground into smaller particles.

For the avoidance of doubt, “treatment” includes the therapeutic treatment, as well as the prophylaxis, of a condition.

The presence of additional substances in a sample, like pharmaceutical excipients, to be characterised by X-ray powder diffraction can mask some of the peaks in the above characterized crystal modification B. This fact alone can of course not demonstrate that the crystal modification B is not present in the sample. Under such circumstances due care must be used and the presence of substantially all main peaks in the X-ray powder diffraction pattern might suffice to characterize the crystal modification B. It is thus preferred to analyse the crystal modifications of the present invention without the presence of additional substances.

According to a further aspect of the invention there is provided a method of treatment of a condition where 4-(5-{(1R)-1-[5-(3-chlorophenyl)isoxazol-3-yl]ethoxy}-4-methyl-4H-1,2,4-triazol-3-yl)pyridine, modification B, is required or desired, which method includes administering a therapeutically effective amount of the crystal modification B according to the present invention to a patient in need of such treatment.

The crystal modification B according to the present invention has the advantage that it is in a form that provides for increased chemical and physical stability, lower hygroscopicity, higher purity, better yield and robust handling properties during manufacturing and post processing, compared to the amorphous form. The present crystal modification B has a well-defined melting point of 141° C. which is approximately 20° C. higher than any other known crystal modification. The skilled person will appreciate that factors such as purity and presence of solvents may influence the melting point.

The crystal form that crystallizes is related to the kinetics and equilibrium conditions of the respective crystal modification at the specific conditions. Thus, as may be appreciated by the skilled person, the crystal modification that is obtained depends upon both the kinetics and the thermodynamics of the crystallization process. Under certain thermodynamic conditions (solvent systems, temperature, pressure and concentration of compound of the invention), one crystal modification may be more stable than another (or indeed any other). However, crystal modifications that have a relatively low thermodynamic stability may be kinetically favoured. Thus, in addition, kinetic factors, such as time, impurity profile, agitation, the presence or absence of seeds, etc may also influence which crystal modification that crystallizes.

The terms “pure”and “pure crystallized fractions” as disclosed herein, relates to 4-(5-{(1R)-1-[5-(3-chlorophenyl)isoxazol-3-yl]ethoxy}-4-methyl-4H-1,2,4-triazol-3-yl)pyridine, modification B, having a purity of at least 90% (wt).

The invention is illustrated, but in no way limited, by the following examples.

### EXAMPLES

### General

X-ray powder diffraction analysis (XRPD) was performed on samples prepared according to standard methods, for example those described in Giacovazzo, C. et al (1995), Fundamentals of Crystallography, Oxford University Press; Jenkins, R. and Snyder, R. L. (1996), Introduction to X-Ray Powder Diffractometry, John Wiley & Sons, New York; Bunn, C. W. (1948), Chemical Crystallography, Clarendon Press, London; or Klug, H. P. & Alexander, L. E. (1974), X-ray Diffraction Procedures, John Wiley and Sons, New York. X-ray analyses were performed using a PANalytical X'Pert Pro, Bragg-Brentano, θ-θ, Cu K_{α}, rotating sample.

XRPD distance values may vary in the range ±2 on the last decimal place.

It will be appreciated by the skilled person that XRPD intensities may vary when measured for essentially the same crystalline form for a variety of reasons including, for example, preferred orientation.

### Reference Example 1

### Preparation of 4-(5-{(1R)-1-[5-(3-chlorophenyl)isoxazol-3-yl]ethoxy}-4-methyl-4H-1,2,4-triazol-3-yl)pyridine, Modification A

(R)-1-[5-(3-chloro-phenyl)-isoxazol-3-yl]-ethanol and 4-(5-methanesulfonyl-4-methyl-4H-[1,2,4]triazol-3-yl) pyridine were obtained in accordance with the disclosure of WO2007/043939. 10 g (44.7 mmol) (R)-1-[5-(3-chloro-phenyl)-isoxazol-3-yl]-ethanol, 12.8 g (53.7 mmol) 4-(5-methanesulfonyl-4-methyl-4H-[1,2,4]triazol-3-yl) pyridine, and 14.6 g (44.7 mmol) caesium carbonate were dissolved/suspended in 50 ml anhydrous dimethylsulfoxide (DMSO). The mixture was heated to and kept at 60° C. during 20 h. The mixture was then heated to 70° C. and additional 2.9 g (8.9 mmol) caesium carbonate was added. After 5.5 h, the conversion was 97%. The mixture was cooled to room temperature while 210 ml water was added to the mixture during 14 h, which generated a phase separation into a liquid and an oil phase. The mixture was then mixed with 100 ml methyl tert-butyl ether, 50 ml isopropyl acetate and 30 ml ethyl acetate which generated two clear liquid phases that were separated. The organic phase was evaporated slowly after which the product crystallized. It was then washed twice with water and isolated. 12.8 g product, corresponding to an isolated yield of 75% was achieved.

### Example 1

### Preparation of 4-(5-{(1R)-1-[5-(3-chlorophenyl)isoxazol-3-yl]ethoxy}-4-methyl-4H-1,2,4-triazol-3-yl)pyridine, Modification B

(R)-1-[5-(3-chloro-phenyl)-isoxazol-3-yl]-ethanol and 4-(5-methanesulfonyl-4-methyl-4H-[1,2,4]triazol-3-yl) pyridine were obtained in accordance with the disclosure of WO2007/043939. 130 g (518.2 mmol) (R)-1-[5-(3-chloro-phenyl)-isoxazol-3-yl]-ethanol and 166.2 g (697.5 mmol) 4-(5-methanesulfonyl-4-methyl-4H-[1,2,4]triazol-3-yl) pyridine were suspended in 650 ml anhydrous dimethyl sulfoxide (DMSO). 189.4 g (581.2 mmol) caesium chloride was added and the mixture was heated to 70° C. The reaction was kept at 70° C. under vigorous stirring for 18 hours after which a conversion of 98.5% had been achieved. The reaction temperature was then adjusted to 20° C. after which 91 ml water was added during 30 min. At this point, the crystallization was initiated by addition of seed crystals (130 mg). The slurry was then kept at 20° C. for 1 h after which additional water (559 ml) was added over 4 h. The mixture was then kept under stirring at 20° C. overnight after which crystals were filtered off and washed twice with DMSO/water (1/1) and twice with water. Finally the crystals were dried at 50° C. under reduced pressure. 207.7 g product corresponding to an isolated yield of 91% was isolated.

### Example 2

### Preparation of 4-(5-{(1R)-1-[5-(3-chlorophenyl)isoxazol-3-yl]ethoxy}-4-methyl-4H-1,2,4-triazol-3-yl)pyridine, Modification B

4 kg (17.9 mol) (R)-1-[5-(3-chloro-phenyl)-isoxazol-3-yl]-ethanol and 5.1 kg (21.4 mol) 4-(5-methanesulfonyl-4-methyl-4H-[1,2,4]triazol-3-yl) pyridine, and 14.6 g (44.7 mmol) were suspended in 22 kg anhydrous dimethylsulfoxide (DMSO). 5.9 kg (18.1 mol) caesium carbonate was added and the mixture was heated to 70° C. The reaction was kept at 70° C. under vigorous stirring overnight after which additional 1.2 kg (3.7 mol) caesium chloride was added. The reaction was kept at 70° C. for 2 h after which the conversion was >99%. The reaction was then clear-filtered and the reactor/filter was rinsed with 2×4.4 kg DMSO. The temperature of the reaction mixture was then ramped from 70° C. to 20° C. over 1 h. 4.0 kg water was added over 1 h to initiate crystallization after which the mixture was left under continuous stirring for 1 h. 24.2 kg water was further added over 4 h. The crystal mixture was then kept under stirring for 8 h. The crystals were filtered off and washed 6 times with DMSO:water (1:1) and 2 times with water. Finally, the crystals were dried at 40° C. under reduced pressure. 5.8 kg product corresponding to an isolated yield of 84% was isolated.

### Example 3

### Preparation of 4-(5-{(1R)-1-[5-(3-chlorophenyl)isoxazol-3-yl]ethoxy}-4-methyl-4H-1,2,4-triazol-3-yl)pyridine, Modification B

4-(5-{(1R)-1-[5-(3-chlorophenyl)isoxazol-3-yl]ethoxy}-4-methyl-4H-1,2,4-triazol-3-yl)pyridine modification A (obtained according to reference example 1) was suspended in ethyl acetate at a temperature of 20° C. for at least 1 h. Crystals of 4-(5-{(1R)-1-[5-(3-chlorophenyl)isoxazol-3-yl]ethoxy}-4-methyl-4H-1,2,4-triazol-3-yl)pyridine, modification B, were recovered.

A similar result was obtained when 4-(5-{(1R)-1-[5-(3-chlorophenyl)isoxazol-3-yl]ethoxy}-4-methyl-4H-1,2,4-triazol-3-yl)pyridine modification A was suspended in 2-propanol at a temperature of 20° C. for at least 1 h.

### Example 4

### X-ray Powder Diffraction (XRPD) Pattern of 4-(5-{(1R)-1-[5-(3-chlorophenyl)isoxazol-3-yl]ethoxy}-4-methyl-4H-1,2,4-triazol-3-yl)pyridine, Modification B

The crystallized fractions obtained in examples 1-3 showed to be pure 4-(5-{(1R)-1-[5-(3-chlorophenyl)isoxazol-3-yl]ethoxy}-4-methyl-4H-1,2,4-triazol-3-yl)pyridine, modification B. Modification B may be identified by the X-ray power diffraction (XPRD) pattern in the table below as well as in FIG. 1.

| d-spacing value (Å) | Relative intensity |
|---|---|
| 10.7 | Very weak |
| 7.6 | Very strong |
| 6.8 | Medium |
| 5.6 | Very strong |
| 4.15 | Strong |
| 4.11 | Medium |
| 3.83 | Medium |
| 3.76 | Medium |
| 3.58 | Weak |

d-value: the spacing between successive parallel hkl planes in a crystal lattice

The peaks, identified with d-values calculated from the Bragg formula and intensities, have been extracted from the diffractogram of 4-(5-{(1R)-1-[5-(3-chlorophenyl)isoxazol-3-yl]ethoxy}-4-methyl-4H-1,2,4-triazol-3-yl)pyridine, modification B, shown in FIG. 1. The relative intensities are less reliable and instead of numerical values the following definitions are used:

| % Relative Intensity* | Definition |
|---|---|
| 25-100 | Very strong |
| 10-25 | Strong |
| 3-10 | Medium |
| 1-3 | Weak |
| <1 | Very weak |

*The relative intensities are derived from diffractograms measured with variable slits.

1. 4-(5-{(1R)-1-[5-(3-chlorophenyl)isoxazol-3-yl]ethoxy}-4-methyl-4H-1,2,4-triazol-3-yl)pyridine in crystalline form.

2. The crystalline form according to claim 1, consisting essentially of the form modification B.

3. 4-(5-{(1R)-1-[5-(3-chlorophenyl)isoxazol-3-yl]ethoxy}-4-methyl-4H-1,2,4-triazol-3-yl)pyridine in crystalline form according to claim 2, characterized in providing an X-ray powder diffraction pattern exhibiting substantially the following main peak with d-values:
d-spacing value (Å)
7.6
5.6

4. 4-(5-{(1R)-1-[5-(3-chlorophenyl)isoxazol-3-yl]ethoxy}-4-methyl-4H-1,2,4-triazol-3-yl)pyridine in crystalline form according to claim 2, characterized in providing an X-ray powder diffraction pattern exhibiting substantially the following main peak with d-values:
d-spacing value (Å)
7.6
6.8
5.6
4.15

5. 4-(5-{(1R)-1-[5-(3-chlorophenyl)isoxazol-3-yl]ethoxy}-4-methyl-4H-1,2,4-triazol-3-yl)pyridine in crystalline form according to claim 2, characterized in providing an X-ray powder diffraction pattern exhibiting substantially the following main peak with d-values:
d-spacing value (Å)
10.7
7.6
6.8
5.6
4.15
4.11
3.83
3.76
3.58

6. 4-(5-{(1R)-1-[5-(3-chlorophenyl)isoxazol-3-yl]ethoxy}-4-methyl-4H-1,2,4-triazol-3-yl)pyridine in crystalline form as defined in claim 2, characterized in providing an X-ray powder diffraction pattern essentially as shown in FIG. 1.

7. A pharmaceutical formulation comprising the compound according to claim 2 in admixture with at least one pharmaceutically acceptable excipient.

8. A method of treatment or prevention of a mGluR5 receptor-mediated disorder selected from the group of gastroesophageal reflux disease, IBS, functional dyspepsia, cough, obesity, Alzheimer's disease, senile dementia, AIDS-induced dementia, Parkinson's disease, amyotrophic lateral sclerosis, Huntington's Chorea, migraine, epilepsy, schizophrenia, depression, anxiety, acute anxiety, obsessive compulsive disorder, ophtalmological disorders such as retinopathies, diabetic retinopathies, glaucoma, auditory neuropathic disorders such as tinnitus, chemotherapy-induced neuropathies, post-herpetic neuralgia and trigeminal neuralgia, tolerance, dependency, addiction and craving disorders, neurodevelopmental disorders including Fragile X, autism, mental retardation, schizophrenia and Down's Syndrome, pain related to migraine, inflammatory pain, chronic pain disorders, acute pain disorders, neuropathic pain disorders such as diabetic neuropathies, arthritis and rheumatitiod diseases, low back pain, post-operative pain, pain associated with various conditions including angina, renal or billiary colic, menstruation, migraine and gout, stroke, head trauma, anoxic and ischemic injuries, hypoglycemia, cardiovascular diseases and epilepsy, which comprises administration of a therapeutically effective amount of a compound according to claim 2, to a patient suffering therefrom.

9. A process for preparing crystalline 4-(5-{(1R)-1-[5-(3-chlorophenyl)isoxazol-3-yl]ethoxy}-4-methyl-4H-1,2,4-triazol-3-yl)pyridine according to claim 2, comprising the steps of:
a) mixing (R)-1-[5-(3-chloro-phenyl)-isoxazol-3-yl]-ethanol, 4-(5-methanesulfonyl-4-methyl-4H-[1,2,4]triazol-3-yl)pyridine and a base in a non-aqueous polar solvent;
b) heating the mixture to at least 60° C. for at least 10 hours;
c) cooling the reaction mixture to a temperature of at most 25° C.; and
d) adding water to the cooled reaction mixture, optionally together with crystalline 4-(5-{(1R)-1-[5-(3-chlorophenyl)isoxazol-3-yl]ethoxy}-4-methyl-4H-1,2,4-triazol-3-yl)pyridine according to claim 2, as seed crystals.

10. A process according to claim 9, characterized in that the non-aqueous polar solvent is selected from the group of dimethylsulfoxide, dimethylformamide, N-methyl pyrrolidone and acetonitrile.

11. A process according to claim 9, characterized in that the base is selected from the group of caesium carbonate and potassium tert-butoxide.

12. A process for preparing crystalline 4-(5-{(1R)-1-[5-(3-chlorophenyl)isoxazol-3-yl]ethoxy}-4-methyl-4H-1,2,4-triazol-3-yl)pyridine according to claim 2, characterized in that crystalline or amorphous 4-(5-{(1R)-1-[5-(3-chlorophenyl)isoxazol-3-yl]ethoxy}-4-methyl-4-H-1,2,4-triazol-3-yl)pyridine is suspended in a solvent chosen from the group of ethyl acetate or 2-propanol at a temperature of at most 20° C. for at least 1 h.
